(19) United States
(12) Patent Application Publication
(10) Pub. No.: US 2007/0027121 A1
(43) Pub. Date: Feb. 1, 2007

(54) Method of treatment of diarrhea-predominant IBS in a female subject receiving contraceptive therapy

(76) Inventors: Steven David Caras, Marietta, GA (US); Troy ZumBrunnen, Marietta, GA (US)

Correspondence Address: MAYER, BROWN, ROWE & MAW LLP, 1909 K STREET, N.W., WASHINGTON, DC, 20006, US

(21) Appl. No.: 11/361,005

(22) Filed: Feb. 24, 2006

Related U.S. Application Data
(60) Provisional application No. 60/656,095, filed on Feb. 25, 2005.
(60) Provisional application No. 60/656,094, filed on Feb. 25, 2005.
(60) Provisional application No. 60/656,103, filed on Feb. 25, 2005.

(51) Int. Cl. A61K 31/56; A61K 31/4184

(52) U.S. Cl. 514/170; 514/394

(57) ABSTRACT

A method of treatment of symptoms associated with diarrhea-predominant IBS and nonconstipated IBS in a female subject receiving contraceptive therapy, which comprises administering a physiologically effective amount of cilansetron or a pharmaceutically acceptable derivative thereof.

### CROSS REFERENCE TO RELATED APPLICATIONS

This application claims the benefit of U.S. Provisional Application Ser. No. 60/656,095, U.S. Provisional Application Ser. No. 60/656,094 and U.S. Provisional Application Ser. No. 60/656,103, all filed Feb. 25, 2005. These applications, in their entirety, are incorporated herein by reference.

### FIELD OF THE INVENTION

The invention relates to the use of cilansetron and, more particularly, to a method for treating diarrhea-predominant irritable bowel syndrome in a female subject receiving contraceptive therapy.

### BACKGROUND OF THE INVENTION

Diarrhea-predominant irritable bowel syndrome (IBS-D) is a condition that is particularly prevalent among women, and which is most commonly diagnosed in women between the ages of 20 and 50 years old. A large number of women in this age group take contraceptives.

While some 5-HT₃ receptor antagonists have shown great promise in treating IBS-D in women, particular 5-HT₃ receptor antagonists have been found to interfere with and adversely affect the performance of contraceptives. For example, Manzo et al, Effect of Alosetron on Pharmacokinetics of Levenorgestrel and Ethinyl Estradiol. AAPS Pharm Sci 4, Abstract (1999) states that co-administration of alosetron with an oral contraceptive containing 30 mg ethinyl estradiol (EE) and 150 mg levonorgestrel (LN) causes 9% and 13% decreases in systemic exposure to the EE and LN components, respectively. This adverse interaction between 5-HT₃ receptor antagonists and oral contraceptives can be caused by any of several factors, including the fact that 5-HT₃ receptor antagonists can induce the metabolism of oral contraceptive components by binding to cytochrome P450 (CYP) enzymes, and the fact that both 5-HT₃ receptor antagonists, as well as the oral contraceptive components have mechanisms of action that are at least partially based on serotonin pathways and may compete to act through these pathways.

Accordingly, there remains a need for an improved method of treatment of IBS-D in women receiving contraceptive therapy.

### SUMMARY OF THE INVENTION

The present invention relates to a method of treatment of symptoms associated with diarrhea-predominant IBS and nonconstipated IBS in a female subject receiving contraceptive therapy, which comprises administering a physiologically effective amount of cilansetron or a pharmaceutically acceptable derivative thereof. Administration of cilansetron or a pharmaceutically acceptable derivative thereof to a female subject receiving contraceptive therapy has been shown herein to have substantially no or no effect on the pharmacokinetic and pharmacodynamic profiles and parameters associated with normal contraceptive use.

### BRIEF DESCRIPTION OF THE DRAWINGS

FIG. 1 is a graph of the mean plasma concentration-time profile for ethinyl estradiol (EE) in two treatment groups.

FIG. 2 is a graph of the mean plasma concentration-time profile for 17-d-norgestimate (17-d-NGM) in two treatment groups.

FIG. 3 is a graph of the mean plasma concentration-time profile for norgestrel (NGL) in two treatment groups.

FIG. 4 is a graph of the mean serum concentration-time profile for follicle stimulating hormone (FSH) in two treatment groups.

FIG. 5 is a graph of the mean serum concentration-time profile for luteinizing hormone (LH) in two treatment groups.

FIG. 6 is a graph of the mean serum concentration-time profile for progesterone (P) in two treatment groups.

### DESCRIPTION OF THE INVENTION

As used herein, “contraceptive” and “contraceptive therapy” are understood to refer to any composition, compound, agent or combination thereof (e.g., in the form of a pill, tablet, capsule, patch, cream, lotion, or any other delivery system) that contain an estrogen component (or synthetic equivalent thereof) and/or a progesterone component (or synthetic equivalent thereof). Non-limiting examples of estrogen components include ethinyl estradiol, esterfied estrogens, estradiol and mestranol. Non-limiting examples of progesterone components include progestins, such as norgestimate norethindrone, norethindrone acetate, desogestrel, drospirenone, ethylnodiol diacetate, norelgestromin, levonorgesrel or dl-norgestrel. The contraceptive or contraceptive therapy, in this regard, can contain any relative amount of estrogen and progesterone components, such that, for example, the contraceptive or contraceptive therapy is monophasic, biphasic or triphasic. Moreover, the contraceptive or contraceptive therapy can be estrogen-dominant, progestin-dominant or androgenic. It is also suitable for the contraceptive therapy to contain at least one progestin alone.

“Cilansetron”, as used herein, is understood to refer to (R)-(−)-4,5,6,8,9,10-hexahydro-10-[(2-methyl-1H-imidazol-1-yl)methyl]-11H-pyrido-[3,2,1-jk]-carbazol-11-one (alternative name: (10R)-5,6,9,10-tetrahydro-10-[(2-methyl-1H-imidazol-1-yl)methyl]4H-pyrido [3,2,1-jk]carbazol-11(8H)-one), which is disclosed, for example, in U.S. Pat. No. 6,566,369, the contents of which are incorporated herein by reference.

The present invention provides a method for treatment of diarrhea-predominant IBS (“IBS-D”) or nonconstipated IBS in a female subject receiving contraceptive therapy, which comprises administering a physiologically effective amount of cilansetron or a pharmaceutically acceptable derivative thereof (e.g., acid addition salts and/or solvates thereof), wherein the treatment has substantially no or no effect on the performance of the contraceptive therapy. In another aspect, the present invention may provide a method for treatment of IBS-D or nonconstipated IBS in a female subject receiving contraceptive therapy, which comprises administering a non-contraceptive inhibiting amount, or a non-contraceptive interacting amount, or a non-contraceptive affecting amount of cilansetron or a pharmaceutically acceptable derivative thereof, wherein the treatment has substantially no or no effect on the performance of the contraceptive therapy. For example, in one method of the present invention, administration of cilansetron or a pharmaceutically acceptable derivative thereof to a female subject receiving contraceptive therapy results in substantially no or no change in the pharmacokinetic and/or pharmacodynamic profiles and parameters associated with contraceptive use alone. In another method of the present invention, administration of cilansetron or a pharmaceutically acceptable derivative thereof to a female receiving contraceptive therapy comprising an estrogen component and a progesterone component results in substantially no or no change in the mean plasma concentrations of the estrogen component, progesterone component and/or metabolites thereof, and in substantially no or no change in the mean serum concentrations of one or more endogenous hormones in the female subject (such as follicle stimulating hormone, luteinizing hormone, and/or progesterone), as compared with contraceptive use alone.

In another aspect, administration of cilansetron or a pharmaceutically acceptable derivative thereof to a female receiving contraceptive therapy comprising an estrogen component or synthetic equivalent thereof results in a difference in mean plasma concentration of the estrogen component, synthetic equivalents thereof and/or metabolites thereof in the female subject, as compared with contraceptive use alone, of less than about 25%, less than about 20%, less than about 15%, less than about 10%, less than about 5%, or even less than about 1%. Alternatively, or in addition, administration of cilansetron or a pharmaceutically acceptable derivative thereof to a female receiving contraceptive therapy comprising an estrogen component or synthetic equivalent thereof results in a difference in mean plasma concentration of the estrogen component, synthetic equivalents thereof and/or metabolites thereof in the female subject, as compared with contraceptive use alone, of less than about 150 pg/mL, less than about 125 pg/mL, less than about 100 pg/mL, less than about 75 pg/mL, less than about 50 pg/mL, or even less than about 25 pg/mL.

In yet another aspect, administration of cilansetron or a pharmaceutically acceptable derivative thereof to a female receiving contraceptive therapy comprising a progesterone component or synthetic equivalent thereof results in a difference in mean plasma concentration of the progesterone component, synthetic equivalents thereof and/or metabolites in the female subject, as compared with contraceptive use alone, of less than about 25%, less than about 20%, less than about 15%, less than about 10%, less than about 5%, or even less than about 1%. Alternatively, or in addition, administration of cilansetron or a pharmaceutically acceptable derivative thereof to a female receiving contraceptive therapy comprising a progesterone component or synthetic equivalent thereof results in a difference in mean plasma concentration of the progesterone component, synthetic equivalents thereof and/or metabolites thereof in the female subject, as compared with contraceptive use alone, of less than about 150 pg/mL, less than about 125 pg/mL, less than about 100 pg/mL, less than about 75 pg/mL, less than about 50 pg/mL, or even less than about 25 pg/mL.

In another embodiment, administration of cilansetron or a pharmaceutically acceptable derivative thereof to a female receiving contraceptive therapy results in a difference in mean serum concentration of follicle stimulating hormone (FSH) in the female subject, as compared with contraceptive use alone, of less than about 25%, less than about 20%, less than about 15%, less than about 10%, less than about 5%, or even less than about 1%. Alternatively, or in addition, administration of cilansetron or a pharmaceutically acceptable derivative thereof to a female receiving contraceptive therapy results in a difference in mean serum concentration of FSH in the female subject, as compared with contraceptive use alone, of less than about 4 U/L, less than about 3 U/L, less than about 2 U/L, less than about 1.5 U/L, less than about 1 U/L, less than about 0.75 U/L, less than about 0.5 U/L, less than about 0.25 U/L, or even less than about 0.10 U/L.

In another aspect, administration of cilansetron or a pharmaceutically acceptable derivative thereof to a female receiving contraceptive therapy results in a difference in mean serum concentration of luteinizing hormone (LH) in the female subject, as compared with contraceptive use alone, of less than about 25%, less than about 20%, less than about 15%, less than about 10%, less than about 5%, or even less than about 1%. Alternatively, or in addition, administration of cilansetron or a pharmaceutically acceptable derivative thereof to a female receiving contraceptive therapy results in a difference in mean serum concentration of LH in the female subject, as compared with contraceptive use alone, of less than about 4 U/L, less than about 3 U/L, less than about 2 U/L, less than about 1.5 U/L, less than about 1 U/L, less than about 0.75 U/L, less than about 0.5 U/L, less than about 0.25 U/L, or even less than about 0.10 U/L.

In yet another aspect, administration of cilansetron or a pharmaceutically acceptable derivative thereof to a female receiving contraceptive therapy results in a difference in mean serum concentration of progesterone (P) in the female subject, as compared with contraceptive use alone, of less than about 25%, less than about 20%, less than about 15%, less than about 10%, less than about 5%, or even less than about 1%. Alternatively, or in addition, administration of cilansetron or a pharmaceutically acceptable derivative thereof to a female receiving contraceptive therapy results in a difference in mean serum concentration of P in the female subject, as compared with contraceptive use alone, of less than about 4 U/L, less than about 3 U/L, less than about 2 U/L, less than about 1.5 U/L, less than about 1 U/L, less than about 0.75 U/L, less than about 0.5 U/L, less than about 0.25 U/L, or even less than about 0.10 U/L. In another embodiment, administration of cilansetron or a pharmaceutically acceptable derivative thereof to a female subject receiving contraceptive therapy results in an increase in mean serum concentration of P in the female subject, as compared with contraceptive use alone, of at least 10%, at least 25%, at least 50%, at least 75%, at least 100%, at least 110%, at least 120%, at least 130%, at least 140%, or at least 150%. In yet another embodiment, administration of cilansetron or a pharmaceutically acceptable derivative thereof to a female subject receiving contraceptive therapy results in an increase in mean serum concentration of P in the female subject, as compared with contraceptive use alone, of at least 0.1 ng/mL, at least 0.2 ng/mL, at least 0.4 ng/mL, at least 0.6 ng/mL, at least 0.8 ng/mL, at least 1.0 ng/mL, at least 1.25 ng/mL, at least 1.5 ng/mL, or at least 2.0 ng/mL.

Cilansetron can be administered in any suitable dose, and using any suitable dosing schedule. The dosage of cilansetron administered according to the methods of the present invention may be, for example, from about 0.5 mg to about 16 mg daily, such as from about 1 mg to about 12 mg daily, even from about 2 mg to about 10 mg daily, or from about 4 mg to about 8 mg daily (e.g., about 6 mg daily). Moreover, the dosages may be administered one or more times a day, such as two or more, three or more, or even four or more times daily. In a preferred embodiment, 2 mg cilansetron is administered three times daily (TID). Moreover, as described in U.S. Pat. No. 6,566,369 (the entire contents of which are incorporated herein by reference), for example, cilansetron may be used in the form of a pharmacologically acceptable acid addition salts, such as cilansetron hydrochloride or cilansetron hydrochloride monohydrate. Additionally, cilansetron can be administered in any form and in combination with any known diluent, filler, salt, buffer, stabilizer, solubilizer, lipid, or other material, as disclosed, for example in the '369 patent. In one embodiment, cilansetron is administered in a composition comprising: 4 parts cilansetron hydrochloride monohydrate, 30 parts corn starch, 70 parts lactose, 5 parts Kollidon 25®, 2 parts magnesium stearate, and 3 parts talcum, as described, for example in U.S. Pat. No. 6,566,369 (the entire contents of which are incorporated herein by reference). In another embodiment, cilansetron is administered in a 2 mg film-coated tablet for oral use in humans, consisting essentially of: 2.34 mg cilansetron.HCl.H₂O, 51.78 mg maize starch, 84.48 mg mannitol; 4.90 mg povidone; 0.50 mg citric acid monohydrate; 3.0 5 mg crospovidone; 1.0 mg colloidal anhydrous silica; 2.0 mg stearic acid; and a coating containing Opadry® 03B28686 (white) or Opadry®) Y-1-7000 (white).

In one aspect, the present invention may provide a method for treating symptoms associated with IBS-D or non-constipated IBS (e.g., IBS-D or alternating diarrhea-predominant/constipative IBS) in a female subject receiving contraceptive therapy. For example, the present invention may provide a method for improving bowel habits (e.g., improving stool consistency, improving bowel frequency, decreasing urgency, and/or decreasing bloating) in a female subject receiving contraceptive therapy. Moreover, an aspect of the present invention may provide a method for improving quality of life, such as by decreasing interruption in daily activities, enhancing body image, decreasing food avoidance, enhancing interpersonal relationships, and/or enhancing sexual performance capacity. In another aspect, the present invention may provide a method for treatment of pain and/or discomfort associated with IBS-D or nonconstipative IBS in a female subject receiving contraceptive therapy.

Clinical test data (set forth in the examples set forth below) prove the surprising suitability of cilansetron for the treatment of IBS-D in a female receiving contraceptive therapy.

### EXAMPLES

A double-blind, balanced, randomized, two-period, placebo-controlled, crossover study in 20 healthy female subjects (1845 years of age, and who had received Ortho Tri-Cyclen® oral contraceptive (“OC”) for three or more months immediately preceding the start of the study) of cilansetron administration was conducted. All subjects were to have their OC cycles synchronized so that on study Day 1, they received the first dose of their OC for that cycle. All subjects were to continue to receive daily administrations of OC through each of the two consecutive 28-day study periods. The OC comprised 0.035 mg of ethinyl estradiol (EE) and the following increasing amounts of norgestimate (NGM) on the particular study days indicated: 0.180 mg NGM on Days 1-7; 0.215 mg NGM on Days 8-14; 0.250 mg NGM on Days 15-21; 0.180 mg NGM on Days 29-35; 0.215 mg NGM on Days 3642; and 0.250 mg NGM on Days 4349. In addition, during the first 28 days of the study (“the first period”), half of the subjects were administered 2 mg doses of cilansetron three times daily (TID) for 7 days (i.e., study days 14-21), while the other half of the subjects received equivalent doses of a placebo TID. On the first day of the “second period” of the 56-day study (i.e., day 29), the two groups were crossed over for the administration of the reverse treatment (i.e., those subjects who had been administered cilansetron 2 mg TID in the first period were to receive equivalent placebo administrations, and vice versa) for 7 days (i.e., study days 4249). Thereby, all subjects received both cilansetron and placebo treatments and, therefore, served as their own control. Safety was measured by monitoring adverse events, physical examination, clinical laboratory and pulmonary function tests, vital signs, 12 lead ECG, and telemetry.

Pharmacokinetic Analysis

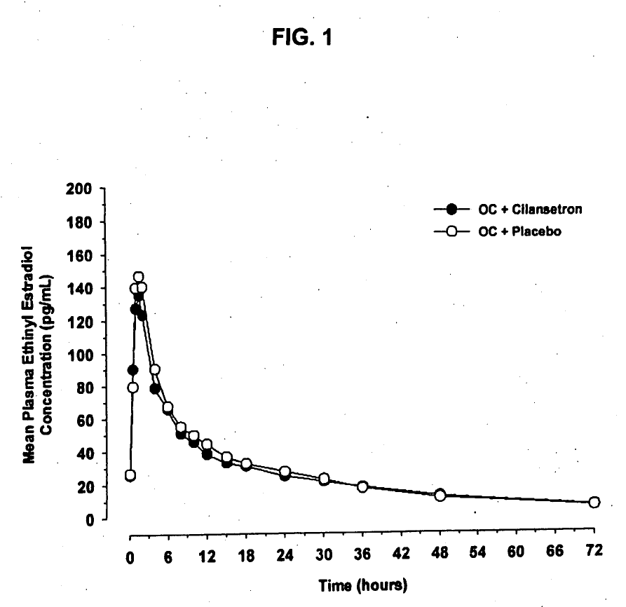

In efforts to determine the effects (if any) of cilansetron 2 mg administration TID for 7 days on the pharmacokinetics (PK) of ethinyl estradiol (EE), norgestimate (NGM), 17-deacetyl-norgestimate (17-d-NGM) and norgestrel (NGL), the mean plasma concentrations of these analytes was determined across the entire time-course of treatment, as set forth in Tables 1-4 and FIGS. 1-3. In addition, the assessed plasma concentrations were used to calculate the AUC₀₋₂₄ (area under the plasma concentration-time curve from time zero to 24 hours), Cₘₐₓ (maximal plasma concentration), Tₘₐₓ (time at which the maximal plasma concentration was observed), λz (terminal elimination rate constant), t_{1/2} (elimination half-life, the time required for the drug plasma concentration to decrease by 50%), and Cₘᵢₙ (minimum plasma concentration), as set forth in Table 5.

Blood Sampling

Ten (10) mL whole blood samples were obtained from each subject for the measurement of EE, NGM, and 17-d-NGM, at the following times during Periods I and II: (i) prior to the morning dose of cilansetron or placebo and OC on Days 1/29, 19/47, 20/48, and 21/49; and (ii) at 0.5, 1, 1.5, 2, 4, 6, 8, 10, 12, 15, 18, 24, 30, 36, 48, and 72 hours after cilansetron and OC or placebo and OC were administered on Days 21/49. Each blood sample was collected in a Vacutainer® tube containing heparin. The blood sample was placed on ice immediately upon collection and centrifuged within 30 minutes. Plasma was separated from red cells by standard centrifugation procedures (4° C., 3,000 rpm for 20 minutes). Plasma was transferred to plastic 10 mL labeled storage tubes immediately following centrifugation. The plasma samples were stored at −20° C. prior to shipment.

Determination of Plasma Concentrations of EE, NGM, 17-d-NGM and NGL

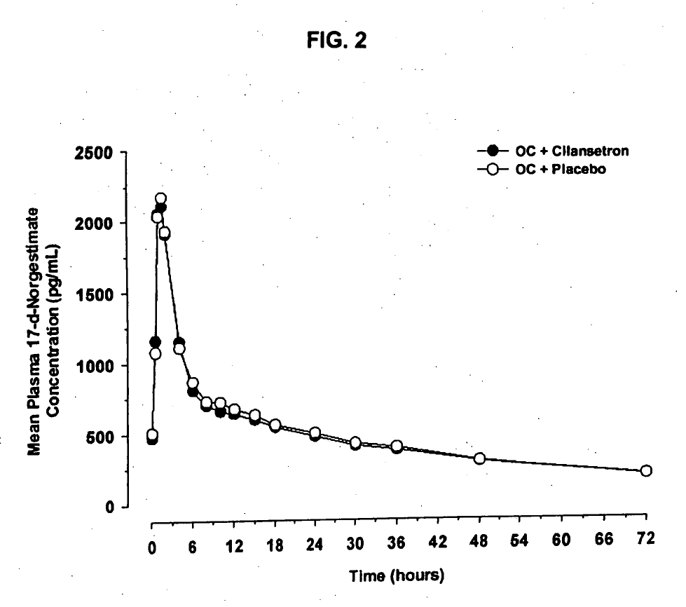

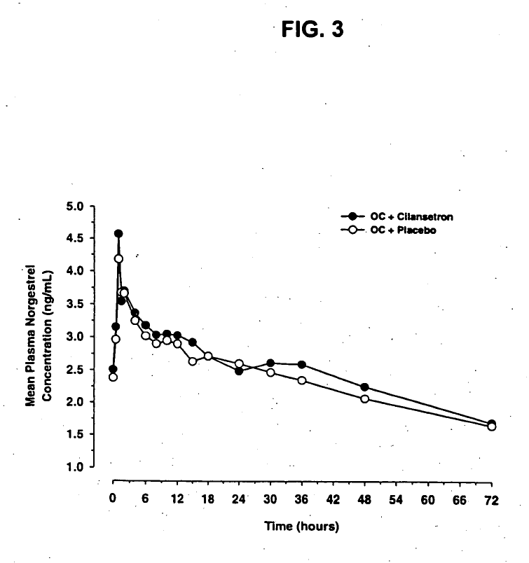

Measurable plasma concentrations of EE, NGM, 17-d-NGM and/or NGL were available for 16 out of the 20 subjects, as determined using validated bioanalytical methods (GC-MS and LC-MS/MS methods). The mean plasma concentration-time profile for EE was determined for both treatment groups in each of the two study periods, and are set forth in Table 1 (the contents of which are graphically illustrated in FIG. 1). The mean plasma concentration-time profile for NGM was determined for both treatment groups in each of the two study periods, and are set forth in Table 2. The mean plasma concentration-time profile for 17-d-NGM was determined for both treatment groups in each of the two study periods, and are set forth in Table 3 (the contents of which are graphically illustrated in FIG. 2). The mean plasma concentration-time profile for NGL was determined for both treatment groups in each of the two study periods, and are set forth in Table 4 (the contents of which are graphically illustrated in FIG. 3).

It is to understood that any ranges, ratios and ranges of ratios that can be formed by any of the numbers or data present herein represent further embodiments of the present invention. This includes ranges that can be formed that do or do not include a finite upper and/or lower boundary. For example, by way of illustration and not limitation, referring to FIG. 1, wherein it is illustrated that the difference in mean plasma concentration of EE between OC+cilansetron and OC administration alone is less than about 10 pg/mL or even less than about 1 pg/mL. Accordingly, the skilled person will appreciate that such ratios, ranges and values are unambiguously derivable from the data presented herein.

TABLE 1

| | Mean Plasma Concentration (pg/mL) of EE at Each Given Time Point | | | | | | | | | |
|---|---|---|---|---|---|---|---|---|---|---|
| Treatment | Day | Day | Day | Day 21/49 | | | | | | |
| Group | 1/29 | 19/47 | 20/48 | predose | 0.5 | 1.0 | 1.5 | 2.0 | 4.0 | 6.0 |
| OC + 2 mg | 0.70 | 26.69 | 23.94 | 25.71 | 90.23 | 127.20 | 135.07 | 123.40 | 78.64 | 65.50 |
| Cilansetron | | | | | | | | | | |
| TID | | | | | | | | | | |
| OC + placebo | 1.89 | 29.78 | 38.59 | 26.44 | 79.58 | 139.52 | 146.43 | 140.01 | 90.43 | 67.38 |

| | Mean Plasma Concentration (pg/mL) of EE at Each Given Time Point | | | | | | | | | |
|---|---|---|---|---|---|---|---|---|---|---|
| Treatment | Day 21/49 | | | | Day 22/50 | | | | Day | Day |
| Group | 8.0 | 10.0 | 12.0 | 15.0 | 18.0 | 24.0 | 30.0 | 36.0 | 23/51 | 24/52 |
| OC + 2 mg | 50.49 | 45.38 | 37.93 | 32.59 | 30.41 | 24.32 | 20.98 | 17.42 | 11.74 | 5.65 |
| Cilansetron | | | | | | | | | | |
| TID | | | | | | | | | | |
| OC + placebo | 54.99 | 49.54 | 44.09 | 36.33 | 32.16 | 26.97 | 22.31 | 16.90 | 10.79 | 5.47 |

TABLE 2

| | Mean Plasma Concentration (pg/mL) of NGM at Each Given Time Point | | | | | | | | | | | | | | | | | | | |
|---|---|---|---|---|---|---|---|---|---|---|---|---|---|---|---|---|---|---|---|---|
| Treatment | Day | Day | Day | Day 21/49 | | | | | | | | | | | Day 22/50 | | | | Day | Day |
| Group | 1/29 | 19/47 | 20/48 | predose | 0.5 | 1.0 | 1.5 | 2.0 | 4.0 | 6.0 | 8.0 | 10.0 | 12.0 | 15.0 | 18.0 | 24.0 | 30.0 | 36.0 | 23/51 | 24/52 |
| OC + 2 mg | 0.0 | 0.0 | 0.0 | 0.0 | 29.0 | 22.5 | 12.9 | 1.5 | 0.0 | 0.0 | 0.0 | 0.0 | 0.0 | 0.0 | 0.0 | 0.0 | 3.1 | 0.0 | 0.0 | 0.0 |
| Cilansetron | | | | | | | | | | | | | | | | | | | | |
| TID | | | | | | | | | | | | | | | | | | | | |
| OC + | 0.0 | 0.0 | 2.3 | 0.0 | 27.4 | 26.7 | 20.9 | 4.8 | 0.0 | 0.0 | 0.0 | 0.0 | 0.0 | 0.0 | 0.0 | 0.0 | 0.0 | 0.0 | 0.0 | 0.0 |
| placebo | | | | | | | | | | | | | | | | | | | | |

TABLE 3

| | Mean Plasma Concentration (pg/mL) of 17-d-NGM at Each Given Time Point | | | | | | | | |
|---|---|---|---|---|---|---|---|---|---|
| Treatment | Day | Day | Day | Day 21/49 | | | | | |
| Group | 1/29 | 19/47 | 20/48 | predose | 0.5 | 1.0 | 1.5 | 2.0 | 4.0 |
| OC + 2 mg | 32.5 | 484.2 | 488.7 | 471.1 | 1155.4 | 2058.6 | 2111.0 | 1910.7 | 1148.0 |
| Cilansetron | | | | | | | | | |
| TID | | | | | | | | | |
| OC + placebo | 6.2 | 503.4 | 537.1 | 506.0 | 1077.6 | 2037.8 | 2176.7 | 1930.0 | 1106.4 |

| | Mean Plasma Concentration (pg/mL) of 17-d-NGM at Each Given Time Point | | | | | | | | | | |
|---|---|---|---|---|---|---|---|---|---|---|---|
| Treatment | Day 21/49 | | | | | Day 22/50 | | | | Day | Day |
| Group | 6.0 | 8.0 | 10.0 | 12.0 | 15.0 | 18.0 | 24.0 | 30.0 | 36.0 | 23/51 | 24/52 |
| OC + 2 mg | 805.4 | 701.6 | 658.5 | 639.6 | 597.3 | 543.2 | 476.5 | 407.7 | 376.4 | 288.0 | 180.1 |
| Cilansetron | | | | | | | | | | | |
| TID | | | | | | | | | | | |
| OC + placebo | 870.1 | 725.9 | 721.7 | 673.7 | 630.4 | 559.3 | 498.1 | 423.8 | 394.0 | 291.8 | 182.2 |

TABLE 4

| | Mean Plasma Concentration (pg/mL) of NGL at Each Given Time Point | | | | | | | | | | |
|---|---|---|---|---|---|---|---|---|---|---|---|
| Treatment | Day | Day | Day | Day 21/49 | | | | | | | |
| Group | 1/29 | 19/47 | 20/48 | predose | 0.5 | 1.0 | 1.5 | 2.0 | 4.0 | 6.0 | 8.0 |
| OC + 2 mg | 250.0 | 2397.9 | 2543.8 | 2503.3 | 3151.3 | 4570.0 | 3544.2 | 3704.3 | 3369.3 | 3179.8 | 3036.7 |
| Cilansetron | | | | | | | | | | | |
| TID | | | | | | | | | | | |
| OC + placebo | 104.1 | 2140.0 | 2454.3 | 2379.1 | 2957.8 | 4180.5 | 3675.9 | 3665.7 | 3248.8 | 3016.5 | 2900.7 |

| | Mean Plasma Concentration (pg/mL) of NGL at Each Given Time Point | | | | | | | | |
|---|---|---|---|---|---|---|---|---|---|
| Treatment | Day 21/49 | | | Day 22/50 | | | | Day | Day |
| Group | 10.0 | 12.0 | 15.0 | 18.0 | 24.0 | 30.0 | 36.0 | 23/51 | 24/52 |
| OC + 2 mg | 3051.8 | 3027.9 | 2924.5 | 2716.2 | 2489.8 | 2614.3 | 2589.6 | 2250.9 | 1697.3 |
| Cilansetron | | | | | | | | | |
| TID | | | | | | | | | |
| OC + placebo | 2948.4 | 2898.1 | 2633.4 | 2718.5 | 2605.6 | 2467.1 | 2347.3 | 2064.9 | 1650.6 |

Table 5 sets forth key descriptive statistics for all pharmacokinetic (PK) parameters (as discussed above) including: AUC(0-24), Cmax, Tmax, λz, t_{1/2}, and Cmin, for the comparison of pharmacokinetic parameters of EE, 17-d-NGM and NGL following multiple oral doses of OC plus placebo and OC plus cilansetron, as discussed above. Statistical analyses of the PK parameters were conducted using an analysis of variance (ANOVA) model with treatment, sequence and period as fixed effects and subject nested within sequence as a random effect, and treatment comparisons for Tmax were done using the Wilcoxon rank sum test.

TABLE 5

| | Mean by Treatment Group | | | | | |
|---|---|---|---|---|---|---|
| | (SD) | | | | | |
| | | | | OC + Placebo | | |
| | OC + Cilansetron | | | (n) | | |
| | (n) | | | | 17-d- | |
| Parameter (Units) | EE | 17-d-NGM | NGL | EE | NGM | NGL |
| AUC (0-24) | 1.209 | 18.897 | 76.429 | 1.323 | 19.47 | 69.732 |
| (hr * ng/mL) | (0.55) | (5.751) | (30.952) | (0.57) | (6.08) | (34.21) |
| Cmax | 0.144 | 2.325 | 5.206 | 0.156 | 2.29 | 4.526 |
| (ng/mL) | (0.06) | (0.891) | (4.519) | (0.06) | (0.79) | (3.87) |
| Tmax | 1.25 | 1.50 | 2.00 | 1.50 | 1.50 | 1.75 |
| (hr) | (1-2) | (1-2) | (1-18) | (1-2) | (1-2) | (1-2) |
| λz | 0.036 | 0.0223 | 0.0146 | 0.035 | 0.022 | 0.0124 |
| (1/hr) | (0.011) | (0.007) | (0.006) | (0.009) | (0.007) | (0.006) |
| t_{1/2} | 20.93 | 33.90 | 55.84 | 21.22 | 34.79 | 67.61 |
| (hr) | (5.072) | (10.56) | (23.41) | (6.065) | (11.85) | (29.75) |
| Cmin | 0.022 | 0.454 | 2.45 | 0.024 | 0.479 | 2.34 |
| (ng/mL) | (0.013) | (0.1634) | (1.01) | (0.013) | (0.163) | (1.19) |

As shown in FIG. 1, the present invention relates to a method for treating IBS in females on OC therapy by administering cilansetron wherein the mean plasma concentration of EE is substantially the same as females receiving OC therapy alone. In another embodiment, the difference in mean plasma concentration of EE between OC+cilansetron and OC therapy alone was no more than about 20%, or no more than about 19%, or no more than about 18%, or no more than about 17%, or no more than about 16%, or no more than about 15%, or no more than about 14%, or no more than about 13%, or no more than about 12%, or no more than about 11%, or no more than about 10%, or no more than about 9%, or no more than about 8%, or no more than about 7%, or no more than about 6%, or no more than about 5%, or no more than about 4%, or no more than about 3%, or no more than about 2%, or no more than about 1%.

As shown in FIG. 2, the present invention relates to a method for treating IBS in females on OC therapy by administering cilansetron wherein the mean plasma concentration of 17-d-NGM is substantially the same as females receiving OC therapy alone. In another embodiment, the difference in mean plasma concentration of 17-d-NGM between OC+cilansetron and OC therapy alone was no more than about 20%, or no more than about 19%, or no more than about 18%, or no more than about 17%, or no more than about 16%, or no more than about 15%, or no more than about 14%, or no more than about 13%, or no more than about 12%, or no more than about 11%, or no more than about 10%, or no more than about 9%, or no more than about 8%, or no more than about 7%, or no more than about 6%, or no more than about 5%, or no more than about 4%, or no more than about 3%, or no more than about 2%, or no more than about 1%.

As shown in FIG. 3, the present invention relates to a method for treating IBS in females on OC therapy by administering cilansetron wherein the mean plasma concentration of norgestrel (NGL) is substantially the same as females receiving OC therapy alone. In another embodiment, the difference in mean plasma concentration of NGL between OC+cilansetron and OC therapy alone was no more than about 20%, or no more than about 19%, or no more than about 18%, or no more than about 17%, or no more than about 16%, or no more than about 15%, or no more than about 14%, or no more than about 13%, or no more than about 12%, or no more than about 11%, or no more than about 10%, or no more than about 9%, or no more than about 8%, or no more than about 7%, or no more than about 6%, or no more than about 5%, or no more than about 4%, or no more than about 3%, or no more than about 2%, or no more than about 1%.

Pharmacodynamic Analysis

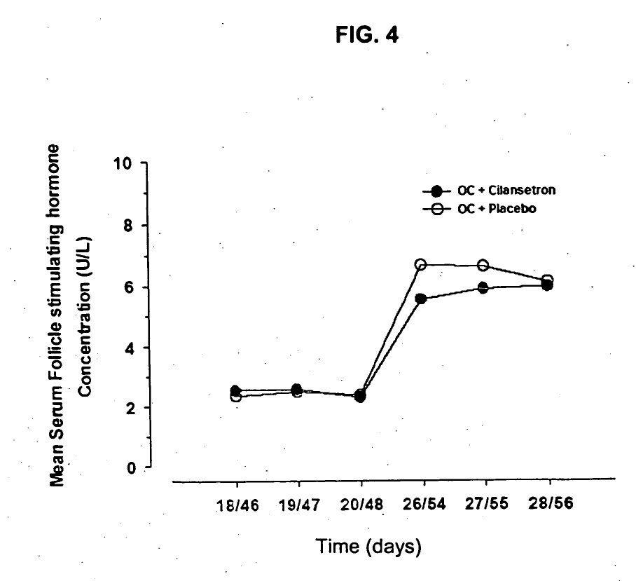

In efforts to determine the effects (if any) of cilansetron 2 mg administration TID for 7 days on the pharmacodynamics of progesterone (P), luteinizing hormone (LH) and follicle stimulating hormone (FSH), the mean serum concentrations of these hormones was determined across the entire time-course of treatment, as set forth in Tables 6-8 and FIGS. 4-6.

Blood Sampling

Whole blood samples (7 mL each) for the measurement of FSH, LH, and P were collected from each subject at the following times during Periods I and II: (i) prior to the morning dose of cilansetron or placebo and OC on Days 18-20 and 46-48; and (ii) prior to the morning dose of OC on Days 26-28 and 54-56. Each 7-mL blood sample was collected in a 7-mL red stopper Vacutainer tube. The blood sample was placed in room temperature to clot. Serum was transferred to plastic 10 mL labeled storage tubes immediately following centrifugation.

Determination of Serum Concentrations of LSH, LH and P

Measurable serum concentrations of LSH, LH and P were available for 17 out of the 20 subjects, as determined using a validated ADVIA Centaur assay. The mean serum concentration-time profile for FSH was determined for both treatment groups in each of the two study periods, and are set forth in Table 6 (the contents of which are graphically illustrated in FIG. 4).

TABLE 6

| | Mean Serum Concentration (U/L) of | | | | | |
|---|---|---|---|---|---|---|
| | FSH at Each Given Time Point | | | | | |
| Treatment | Day | Day | Day | Day | Day | |
| Group | 18/46 | 19/47 | 20/48 | 26/54 | 27/55 | Day 28/56 |
| OC + 2 mg | 2.55 | 2.59 | 2.31 | 5.56 | 5.89 | 5.98 |
| Cilansetron | | | | | | |
| TID | | | | | | |
| OC + placebo | 2.36 | 2.50 | 2.39 | 6.66 | 6.62 | 6.12 |

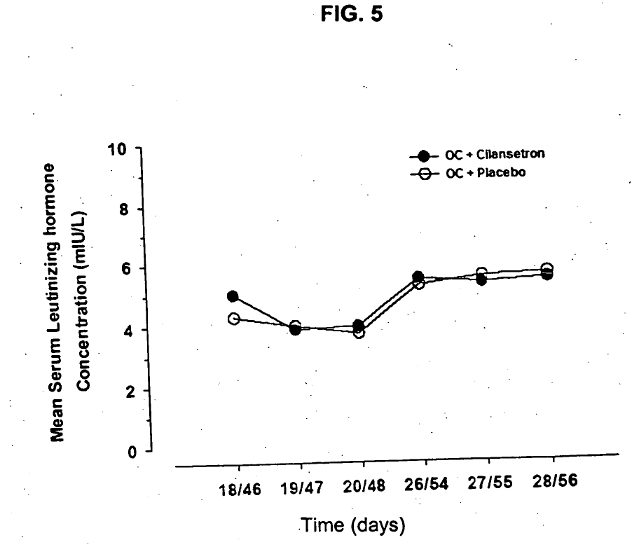

The mean serum concentration-time profile for LH was determined for both treatment groups in each of the two study periods, and are set forth in Table 7 (the contents of which are graphically illustrated in FIG. 5).

TABLE 7

| | Mean Serum Concentration (U/L) of | | | | | |
|---|---|---|---|---|---|---|
| | LH at Each Given Time Point | | | | | |
| Treatment | Day | Day | Day | Day | Day | |
| Group | 18/46 | 19/47 | 20/48 | 26/54 | 27/55 | Day 28/56 |
| OC + 2 mg | 5.03 | 3.89 | 3.98 | 5.48 | 5.34 | 5.47 |
| Cilansetron | | | | | | |
| TID | | | | | | |
| OC + placebo | 4.31 | 4.01 | 3.74 | 5.27 | 5.56 | 5.64 |

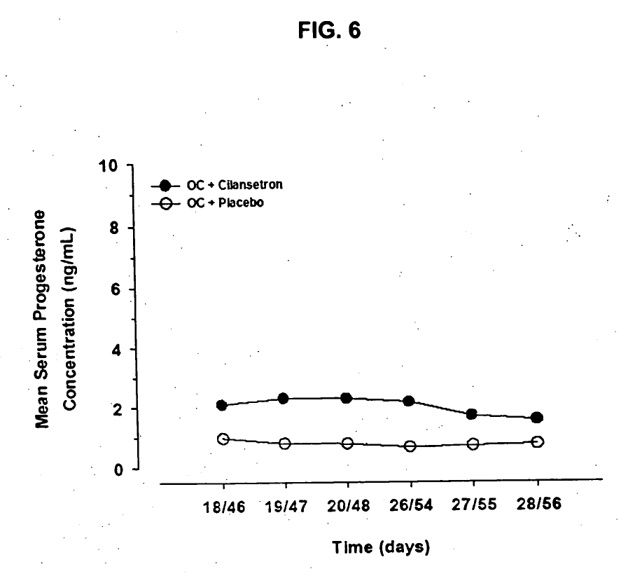

The mean serum concentration-time profile for P was determined for both treatment groups in each of the two study periods, and are set forth in Table 8 (the contents of which are graphically illustrated in FIG. 6).

TABLE 8

| | Mean Serum Concentration (ng/mL) of | | | | | |
|---|---|---|---|---|---|---|
| | P at Each Given Time Point | | | | | |
| Treatment | Day | Day | Day | Day | Day | |
| Group | 18/46 | 19/47 | 20/48 | 26/54 | 27/55 | Day 28/56 |
| OC + 2 mg | 2.10 | 2.31 | 2.29 | 2.16 | 1.70 | 1.57 |
| Cilansetron | | | | | | |
| TID | | | | | | |
| OC + placebo | 0.99 | 0.81 | 0.79 | 0.67 | 0.72 | 0.77 |

Table 9 sets forth key pharmacodynamic descriptive statistics, including least squares (LS) means, LS mean differences, 90% Cls on ratios, and p-values for the comparison of serum concentration levels of FSH, LH and P, following multiple oral doses of OC plus placebo and OC plus cilansetron. Statistical analyses of the pharmacodynamic parameters were conducted using an analysis of variance (ANOVA) model for a 2×2 crossover design with repeated measurements with treatment, sequence and period as fixed effects and subject (sequence) and treatment*subject (sequence) as random effects with the auto-correlation structure defined as spatial power within the period.

TABLE 9

| | | | | Treatment Comparisons | | | |
|---|---|---|---|---|---|---|---|
| | | | | | Difference | | |
| Analyte | | | | | (Test- | 90% CI for | |
| (Units) | Treatment | N | LS Mean | SD | Reference) | Difference | p-Value |
| P | OC + Cilansetron | 17 | 1.99 | 0.77 | 1.16 | (−0.75-3.08) | 0.3031 |
| (ng/mL) | OC + Placebo | 17 | 0.83 | 0.77 | — | — | — |
| LH | OC + Cilansetron | 17 | 4.98 | 0.51 | 0.22 | (−0.79-1.24) | 0.7104 |
| (mIU/mL) | OC + Placebo | 17 | 4.75 | 0.51 | — | — | — |
| FSH | OC + Cilansetron | 17 | 4.17 | 0.47 | −0.15 | (−1.27-0.97) | 0.8198 |
| (U/L) | OC + Placebo | 17 | 4.32 | 0.47 | — | — | — |

As shown in FIG. 4, the present invention relates to a method for treating IBS in females on OC therapy by administering cilansetron wherein the mean serum concentration of LSH is substantially the same as females receiving OC therapy alone. In another embodiment, the difference in mean serum concentration of FSH between OC+cilansetron and OC therapy alone was no more than about 20%, or no more than about 19%, or no more than about 18%, or no more than about 17%, or no more than about 16%, or no more than about 15%, or no more than about 14%, or no more than about 13%, or no more than about 12%, or no more than about 11%, or no more than about 10%, or no more than about 9%, or no more than about 8%, or no more than about 7%, or no more than about 6%, or no more than about 5%, or no more than about 4%, or no more than about 3%, or no more than about 2%, or no more than about 1%.

As shown in FIG. 5, the present invention relates to a method for treating IBS in females on OC therapy by administering cilansetron wherein the mean serum concentration of LH is substantially the same as females receiving OC therapy alone. In another embodiment, the difference in mean serum concentration of LH between OC+cilansetron and OC therapy alone was no more than about 20%, or no more than about 19%, or no more than about 18%, or no more than about 17%, or no more than about 16%, or no more than about 15%, or no more than about 14%, or no more than about 13%, or no more than about 12%, or no more than about 11%, or no more than about 10%, or no more than about 9%, or no more than about 8%, or no more than about 7%, or no more than about 6%, or no more than about 5%, or no more than about 4%, or no more than about 3%, or no more than about 2%, or no more than about 1%.

As shown in FIG. 6, the present invention relates to a method for treating IBS in females on OC therapy by administering cilansetron wherein the mean serum concentration of endogenous progesterone (P) is substantially the same as females receiving OC therapy alone. In another embodiment, the difference in mean serum concentration of P between OC+cilansetron and OC therapy alone was no more than about 20%, or no more than about 19%, or no more than about 18%, or no more than about 17%, or no more than about 16%, or no more than about 15%, or no more than about 14%, or no more than about 13%, or no more than about 12%, or no more than about 11%, or no more than about 10%, or no more than about 9%, or no more than about 8%, or no more than about 7%, or no more than about 6%, or no more than about 5%, or no more than about 4%, or no more than about 3%, or no more than about 2%, or no more than about 1%.

What is claimed is:

1. A method of treatment of diarrhea-predominant IBS in a female subject receiving contraceptive therapy, which comprises administering a physiologically effective amount of cilansetron or a pharmaceutically acceptable derivative thereof.

2. The method of claim 1, wherein said contraceptive therapy comprises an estrogen component or synthetic equivalent thereof and/or a progesterone component or synthetic equivalent thereof.

3. The method of claim 2, wherein said contraceptive therapy comprises an estrogen component or synthetic equivalent thereof.

4. The method of claim 2, wherein said contraceptive therapy comprises a progesterone component or synthetic equivalent thereof.

5. The method of claim 3, wherein said contraceptive therapy comprises ethinyl estradiol.

6. The method of claim 4, wherein said contraceptive therapy comprises a progestin.

7. The method of claim 1, wherein said treatment causes substantially no effect on the performance of the contraceptive therapy.

8. The method of claim 2, wherein said treatment has substantially no effect on the plasma concentration of the estrogen component of the contraceptive therapy in the female subject.

9. The method of claim 2, wherein said treatment has substantially no effect on the mean plasma concentration of the progesterone component of the contraceptive in the female subject.

10. The method of claim 2, wherein said treatment results in a difference in mean plasma concentration of the estrogen component, synthetic equivalents thereof and/or metabolites thereof in the female subject of less than about 15%, as compared with contraceptive use alone.

11. The method of claim 3, wherein said treatment results in a difference in mean plasma concentration of the progesterone component, synthetic equivalents thereof and/or metabolites thereof in the female subject of less than about 15%, as compared with contraceptive use alone.

12. The method of claim 1, wherein said treatment results in a difference in mean serum concentration of follicle stimulating hormone in the female subject of less than about 15%, as compared with contraceptive use alone.

13. The method of claim 1, wherein said treatment results in a difference in mean serum concentration of luteinizing hormone in the female subject of less than about 15%, as compared with contraceptive use alone.

14. The method of claim 1, wherein cilansetron is administered in a dose of 2 mg, three times daily.

15. The method of claim 1, wherein the cilansetron comprises a hydrochloride salt of cilansetron.

16. A method of treatment of diarrhea-predominant IBS in a female subject receiving contraceptive therapy, which comprises administering a non-contraceptive inhibiting amount amount of cilansetron or a pharmaceutically acceptable derivative thereof.

17. A method of treatment of nonconstipated IBS in a female subject receiving contraceptive therapy, which comprises administering a physiologically effective amount of cilansetron or a pharmaceutically acceptable derivative thereof.

18. A method of improving bowel habits in a female subject afflicted with diarrhea-predominant IBS, wherein the female subject is receiving contraceptive therapy, which comprises administering a physiologically effective amount of cilansetron or a pharmaceutically acceptable derivative thereof.

19. A method for improving quality of life for a female subject afflicted with diarrhea-predominant IBS, wherein the female subject is receiving contraceptive therapy, which comprises administering a physiologically effective amount of cilansetron or a pharmaceutically acceptable derivative thereof.

20. A method for treatment of pain associated with diarrhea-predominant IBS in a female subject receiving contraceptive therapy, which comprises administering a physiologically effective amount of cilansetron or a pharmaceutically acceptable derivative thereof.
